(19) United States
(12) Patent Application Publication
(10) Pub. No.: US 2004/0192598 A1
(43) Pub. Date: Sep. 30, 2004

(54) Composition and method of alleviating adverse side effects and/or enhancing efficacy of agents that inhibit aromatase

(76) Inventor: Laura Kragie, Oak Park, IL (US)

Correspondence Address: Pratt & Associates Inc, 10821 Hillbroke Lane, Potomac, MD, 20854, US

(21) Appl. No.: 10/474,838

(22) PCT Filed: Oct. 10, 2001
(86) PCT No.: PCT/US01/32066

Related U.S. Application Data
(60) Provisional application No. 60/239,457, filed on Oct. 11, 2000.

(51) Int. Cl. A61K 48/00; A61K 31/56; A61K 38/22

(52) U.S. Cl. 514/12; 514/44; 514/171

(57) ABSTRACT

This disclosure describes compositions and methods of use of compositions, that can replace the role of estrogens in the functions of humans and other animals, when these humans or animals are under the influence of compounds, devices and biologics that can inhibit the activity of aromatase enzyme (estrogen synthetase).

This application claims priority to provisional application U.S. Serial No. 60/239,457 filed Oct. 11, 2000.

### FIELD

The present invention relates to compositions and methods of use of such compositions to prevent and/or to treat diseases attributed to estrogen deficit resulting from exposure to aromatase inhibition.

### BACKGROUND

Medications, therapies, foods and environmental agents, often inadvertently inhibit the production of endogenous estrogens, leading to their tissue deficit. The consequences of estrogen deprivation in humans and animals include acne, hirsuitism, alopecia, vaginitis, urogenital dysfunction, infertility, pregnancy loss, dysfunctional parturition, cardiovascular disease, lipidemias, vasomotor symptoms, memory dysfunction, motor dysfunction, mood disorders, immune disorders, migraine headaches, osteoporosis, and arthritis. Thus there is a great need to replace or prevent this loss of estrogen in order to ameliorate the signs, symptoms and diseases associated with systemic and local estrogen synthesis inhibition and to improve the overall efficacy of therapeutic regimens.

Aromatase is the key enzyme complex in the biochemical synthetic pathway for estrogen. In primates and other animals testosterone, which is usually produced endogenously from gonadal tissues, is converted by the aromatase enzyme (a.k.a. estrogen synthetase) into estrogens. Aromatase is an enzyme-complex involving NADPH-cytochrome C reductase and a specific cytochrome P-450 protein (gene product of CYP19 or P450arom). The reaction which is catalyzed by aromatase is unique in the biosynthesis of steroids, as it involves conversion of ring A of the steroid structure to an aromatic ring with the loss of the angular C-19 methyl group and the cis-elimination of the 1beta and 2beta hydrogens to yield estrogen and formic acid.

Androgens and other estrogen precursors are also produced in peripheral tissues. Primate adrenals secrete large amounts of the precursor steroid dehydroepiandrosterone (DHEA) and especially DHEA-sulfate (DHEA-S), which are converted into androstenedione or androstenediol and then into potent androgens and estrogens in the peripheral tissues. Androstenedione is the precursor of estrone, which is a main source of the potent and biologically active estrogen, estradiol, in postmenopausal women. DHEA-S, the major steroid present in blood of both men and women, is converted into DHEA and androstenediol in peripheral tissues. Depending upon the relative activities of 17beta-hydroxysteroid dehydrogenase, aromatase and 5alpha-reductase, DHEA or its derivatives will be preferentially converted into androgens and/or estrogens (Adams J B. Mol Cell Endocrinol 1985; 41:1-17; Labrie F. J Endocrinol Invest 1998; 21:399-401; Labrie F, et al. J Clin Endocrinol Metab 1997; 82:3498-505).

Aromatase is present in ovarian and testicular cells but also in various extra-gonadal tissues. Target tissues possess the enzymatic machinery necessary to synthesize androgens and/or estrogens according to local control and need. For example, the skin is an important site of sex steroid formation (Labrie F. J Endocrinol Invest 1998; 21:399-401; Labrie F, et al. J Clin Endocrinol Metab 1997; 82:3498-505). Studies in rat show that estrogen is produced locally in vaginal tissues by aromatase (Lephart E D, et al. Biol Reprod 1989; 40:259-67). Aromatase is tissue-specifically regulated by various factors. This tissue-specific regulation of human aromatase gene is realized by alternative splicing of multiple exons that are tissue specific promotors for expression in the placenta, skin fibroblasts, fetal liver, ovary, prostate, testis, placenta, and brain. Evidence indicates that estrogen, locally-produced by aromatase, acts in various tissues as a multi-functional paracrine or autocrine hormone: (i) aromatase is distributed in various gonadal and extra-gonadal tissues, (ii) aromatase is regulated tissue-specifically by various factors, (iii) the aromatase product estrogen participates in specific physiological functions of various tissues, and (iv) estrogen receptors are distributed in various tissues (Harada N. Nippon Yakurigaku Zasshi 1998; 112:51-8).

Estrogen has atheroprotective effects and there are estrogen receptors present in vascular structures. Aromatase is also found in human vascular smooth muscle cells (SMCs) using in situ hybridization technique. These findings suggest that estrogen is synthesized locally and then directly acts in an autocrine or paracrine manner, with possible cross talk between smooth muscle and endothelial cells (Harada N, et al. Circ Res 1999; 84:1285-91; Dandona P, et al. Endocrine Soc 77th Ann Mtg, 1995; Hayashi T, et al. Arterioscler Thromb Vasc Biol 2000; 20:782-92; Rosenfeld C R, et al. Am J Physiol Heart Circ Physiol 2000; 279:H319-28).

Cessation of ovarian estrogen secretion is the key event during the climacteric. Aromatase expression in adipose tissue and skin primarily accounts for the extraglandular or peripheral formation of estrogen. Aromatase activity increases as a function of body weight and advancing age. Sufficient circulating concentrations of the biologically active estrogen, estradiol, can be produced as a result of extraglandular aromatization of androstenedione to estrone, which is subsequently reduced to estradiol in peripheral tissues. Extraglandular aromatase expression in adipose tissue and skin (via the increase in circulating levels of estradiol) and in bone (via increasing local estrogen concentrations) is important in slowing the rate of postmenopausal bone loss. Whether systemically delivered or locally produced, elevated estrogen concentrations promote the growth of these steroid-responsive tissues. Local biosynthesis of estrogen by aromatase in the brain may be important in the regulation of various cognitive and hypothalamic functions (Bulun S E, et al. Semin Reprod Endocrinol 1999; 17:349-58; Cyr M, et al. Curr Pharm Des 2000; 6:1287-312).

Estrogen synthesis can be blocked specifically by inhibiting aromatase. Aromatization is the last and critical step in the biosynthesis of estrogens from cholesterol. Therefore, specific blockade of this enzyme does not cause deprivation of other essential steroids such as glucocorticoid, mineralocorticoid and androgen steroids. Specific aromatase inhibitors have been used for the treatment of female breast cancer where estrogens stimulate tumor growth, and the aromatase inhibitor deters tumor growth by depleting estrogens. In men, aromatase inhibitors decrease estradiol concentrations and simultaneously increase testosterone concentrations. They have been used to treat prostate cancer and prostate hyperplasia.

Several antifungal pharmaceutical agents exert their therapeutic effect by inhibiting sterol formation in the yeast cell. However, imidazole antifungals also unintentionally inhibit aromatase activity in animals, including humans (Kragie L et al. 10th Intl Congress Endo 1996; #P3-480; Mason J I, et al. Biochemical Pharmacology 1985; 34:1087-92). Antifungal therapeutics are administered to humans and animals through a variety of routes. Vaginal and vulvar topical preparations are used for vaginal and vulvar candidiasis. Finger and toe nail fungal infections are treated with months of daily oral antifungal chemotherapeutic agents. Topical and oral antifungals are given to treat skin fungal infections. Intravenous antifungals are often given to immunocompromised patients such as those with Acquired Immunodeficiency Syndrome, those undergoing cancer chemotherapy or bone marrow transplant or those with selective immunodeficiency syndromes due to hematologic diseases. Patients with fungal meningitis or brain abscesses may be given prolonged parenteral or intrathecal antifungal therapy. Patients with systemic candidemia may also receive intravenous antifungal therapy. Patients with yeast nephritis and cystitis arising from prolonged antibiotic therapy may also receive intravenous antifungal therapy or have antifungal therapy instilled in the bladder as an irrigation or wash.

Neither the US FDA approved patient insert nor physician/pharmacy instructional materials of the US FDA approved antifungal products contain any information indicating that these products inhibit aromatase, nor do they contain any warnings/precautions/restrictions on the use of these approved products that, by inhibiting aromatase, produce estrogen deficit.

As for the aromatase inhibitors used in treating breast and prostate cancers, although the related scientific literature discusses the ability of these agents to reduce plasma estrogen levels, no suggestion is made for any estrogen replacement or “add back” therapies. Nor do they suggest selective-estrogen receptor modulators (SERM) agents as adjuvant therapies to combine with the aromatase inhibition. Only anti-estrogenic therapies are discussed as possible candidates for adjuvant therapy to the aromatase inhibitor, with the intent to further decrease and shut down estrogenic functions within the body. There are no FDA approved therapies that combine an aromatase inhibitor and an estrogenic compound. In fact, the commercially available antifungal vulvovaginal cream and suppository products specifically instruct the consumer to discontinue other vaginal therapy products while administering the antifungal product. The product labels instruct women administering vaginal estrogen creams for the indication hypogonadal vaginitis to discontinue the hormone therapy during the antifungal treatment of vaginal candidiasis. In fact, the product labels for estrogen therapies specifically cite vulvovaginal candidiasis as a side effect of hormone therapy. Thus, the current understanding of the imidazole antifungal products and the current standard of clinical gynecologic practice actually teaches the opposite of the proposed invention. And the current standard approaches to breast cancer treatment that combine aromatase inhibitors with adjuvant estrogen receptor antagonists, also teach away from the invention.

### SUMMARY

Thus, it is one object of this invention to provide a composition comprising estrogen function replacement (EFR) agent(s) that can replace the role of estrogens, such as estradiol, in the functions of humans and animals. These compositions can be administered to humans or animals under the influence of compounds, devices and/or biologics that can inhibit the activity of their aromatase enzyme, estrogen synthetase. An EFR agent, as described in this application, is defined as one that can selectively, totally, or partially replace the functions performed by the estrogen compounds that are usually synthesized by the aromatase enzyme. Compounds and therapeutics which inhibit aromatase activity can be identified using assays described in the scientific literature, such as the placental microsome assay (Kragie L et al. 10th Intl Congress Endo 1996; #P3-480; Mason J I, et al. Biochemical Pharmacology 1985; 34:1087-92) among others.

It is another object of this invention to provide a method of using compositions containing EFR agents, to treat humans or animals when they are under the influence of compounds, devices and/or biologics that can inhibit the activity of their aromatase enzymes. The method comprises administering EFR agent(s) through oral, inhaled, topical, parenteral, rectal, intravaginal, intraurethra, intrathecal or implanted route(s) in combination with the exposure to aromatase inhibitor(s). The EFR agent(s) can be administered simultaneously or disjoint in time, preceding or succeeding the administration of the aromatase inhibitor. The EFR agent(s) can be given for more, less or the same duration as the aromatase inhibitor agent(s).

It is another object of the invention to provide a method for preventing or alleviating adverse side effects and/or enhancing the beneficial efficacy of therapeutic agents that inhibit aromatase.

Additionally, it is another object of this invention to provide a method for treating diseases in humans and animals resulting from the exposure to compounds, devices and biologics that can inhibit the activity of aromatase enzyme.

Specific examples of the invention include, but are not restricted to, combining EFR agents with the intentional (therapeutic) and/or nonintentional exposure to aromatase inhibitors in humans and other animals, such as described in the following:

vaginal, vulvar, inguinal and skin topical antifungal preparations.

oral antifungal agents used for long term treatment of such infections as nail fungal infections, oropharyngeal and esophageal candidiasis, histoplasmosis, blastomycosis, cryptocococus, coccidioides, aspergillus and tuberculosis.

intravenous antifungal agents given to immunocompromised patients, such as those with AIDs, undergoing cancer chemotherapy or bone marrow transplant or those with selective immunodeficiency syndromes and hematologic diseases.

intravenous and intrathecal antifungal agents given to patients with fungal meningitis or brain abscess.

chemotherapies given for breast cancer and for prostate cancer.

psychotropic drugs, such as midazolam, as used in anesthesia, antianxiety and antiepileptic therapies.

contraceptive hormones, such as norethindrone (17 alpha-ethynyl-19-nortestosterone), an irreversible inhibitor of aromatase.

herbal and plant supplements including Over-the-Counter products and prescription botanical products.

tobacco smoke, as occurs in nicotine-addicted subjects and especially pregnant nicotine-addicted subjects.

### DETAILED DESCRIPTION

Aromatase Inhibitors:

The compound(s) that comprise the group, “aromatase inhibitor,” may be any combination of chemical, drug, biologic, device, botanical product, herb supplement, vitamin supplement, dietary supplement, food product, food toxin, bacterial or viral product, air contaminant, water contaminant, or drug contaminant. Administration of the aromatase inhibitor to the human or mammal may be intentional, unintentional, or unavoidable. Prodrugs that are metabolized into a compound with aromatase inhibitory properties, are included in this definition. Prodrug aromatase inhibitor examples include compounds that are acted on in vivo by such enzyme reactions as hydrolysis, (de)hydroxylation, oxidation, reduction, sulfotransferase, (de)methylation, (de)lipidation, (de)prenylation, (de)glycosylation, (de)glucuronidation, (de)acetylation, (de)phosphosphorylation, (de)hydration, encapsulation, digestion and cellular transport. The compound can be a “caged-precursor” which is a chemical structure that undergoes transformation when triggered by a stimulus such as light or bioelectrical activity. The compound may be produced de novo in a protected compartment implanted within the human or animal. The aromatase inhibitor, its stereoisomers and nontoxic pharmacologically acceptable salts, can be administered by various routes. The dosage of the aromatase inhibitor compounds would vary with the particular condition being treated, the severity of the condition, the duration of the treatment, the administration route and the specific compound(s) being employed. If the aromatase inhibitor exposure is nonintentional, such as with tobacco smoke, then the compound's dosage and exposure duration can be assessed to estimate pharmacodynamic effect and thus, the consequential estrogen deficit to be replaced. Inhibitors of aromatase have been developed as pharmaceutical treatments for postmenopausal and estrogen receptor positive breast cancer. Both steroidal substrate analogs (type I) inhibitors, which inactivate the enzyme, and non-steroidal competitive reversible (type II) inhibitors, are available as treatments. 4-hydroxyandrostenedione (4-OHA), one of the earliest marketed selective aromatase inhibitors, is used to reduce blood and tissue estrogen concentrations in patients with hormone responsive disease. Letrozole and anastrozole also are similar such treatments for breast cancer. Both agents suppress serum estrogen levels to the limit of assay detection (Brodie A, et al. J Steroid Biochem Mol Biol 1999; 69:205-10).

Aromatase inhibitors can be identified using the placental aromatase assay as described in (Mason J I, et al. Biochemical Pharmacology 1985; 34:1087-92) or derivations of it, such as those using recombinant enzymes (Stresser D M, et al. Anal Biochem 2000; 284:427-30). The placental microsomal aromatase assay is a convenient and informative screening tool to assess drug interaction with estrogen formation from aromatase activity. The aromatase inhibitor activity, and the concentration range of inhibitor effect, can be identified by dose-response evaluation of the agent in the assays of aromatase enzyme activity. Probable target tissue concentrations of aromatase inhibitor can be estimated by assessing subject's inhibitor exposure and the bioavailability of the aromatase agent at the target site. This data can then be compared to the dose-response information from the aromatase assay and used to predict the probable estrogen deficit resulting from exposure.

Examples of Aromatase Inhibitors Include, but are not Limited to:

Norethisterone, norethindrone, [17alpha-ethynyl-19-nortestosterone] (Osawa Y, Yarborough C. Science 1982; 215:1249-51; Yamamoto, et al. Eur J Endocrinol 1994; 130:634-40); 13-retro-antiprogestins (Shimizu Y, et al. Steroids 1995; 60:234-8); aminoglutethimide and testololactone (Santner S J, et al. J Steroid Biochem 1984; 20:1239-42); anastrozole (Dowsett M, et al. Cancer Chemother Pharmacol 2000; 46:35-9); fadrozole, vorozole, roglethimide, atamestane, exemestane, formestane, YM-511(4-[N-(4-bromobenzyl)-N-(4-cyanophenyl)amino]-4H-1,2,4-triazole), ZD-1033, arimedex, NKS-01, 14-alpha.-hydroxyandrost-4-ene-3,6,17-trione (Santti, et al. U.S. Pat. No. 5,972,921, 1999); ketoconazole, bifonazole, clotrimazole, econazole, isoconazole, miconazole and tioconazole (Ayub M, Levell M J. Biochem Pharmacol 1990; 40:1569-75); voriconazole; midazolam (Kragie L et al. 10th Intl Congress Endo 1996; #P3-480); certain azole derivatives (Hirsch, et al. U.S. Pat. No. 4,755,526 1988); aromatase inhibiting 4(5)-imidazoles and other selective compounds (Karjalainen et al U.S. Pat. No. 5,962,495, 1999; Karjalainen, et al. U.S. Pat. No. 5,098,923, 1992); tobacco leaf, smoke extracts, vegetables, plant leaves and fruits (Osawa Y, et al. J Enzyme Inhib 1990; 4:187-200); the synthetic flavonoid alpha-naphthoflavone; naturally-occurring flavonoids, chrysin, flavone, genistein 4′-methyl ether, Biochanin A (Campbell D R, Kurzer M S. J Steroid Biochem Mol Biol 1993; 46:381-8); insulin sensitizer troglitazone (Mu Y M, et al. Biochem Biophys Res Commun 2000; 271:710-3).

Estrogen Function Replacement Agents

Estrogens are a class of gonadal steroid hormones associated with the development and maintenance of secondary female sex characteristics, control of the cyclical changes in the reproductive cycle, are required for pregnancy maintenance and have an anabolic effect on protein metabolism and water retention. Estrogens have genomic actions that occur through interaction with nuclear estrogen receptors and subsequent gene transcription and expression. Estrogens may also act in nongenomic manners affecting membrane activities, lipid fluidity, metabolism, biochemical reactions (e.g., redox biochemical reactions) and nongenomic estrogen receptor mediated actions (Whiting K P, et al. Life Sci 2000;67:743-57). An EFR agent as described in this application, is defined as one that can selectively, partially, or totally replace the functions of the estrogen compounds, such as estradiol and estrone, that are synthesized from the substrates of the estrogen synthetase/aromatase enzyme, in a human or other animal. The agent(s) may act directly or indirectly through an induced intermediary. The agent(s) may act at the same cellular or molecular branch point as the referenced estrogen, or they may act downstream from that branch point. They may partially or completely replace all of the referenced estrogen functions, a select subset of functions, or only one specific function.

The EFR agent can be any estrogenic agent from any source (e.g., synthetic, plant-derived or animal source) or any selective estrogen receptor agonist or ligand used to selectively stimulate a particular estrogen-associated biological action. The EFR agent could also be a biologic product or medical device that delivers or produces de novo, an agent that performs estrogen function(s) in the body of the human or animal. The function(s) could be directly or indirectly associated with the presence of natural endogenous estrogens synthesized via the aromatase enzyme.

EFR agents include the group defined as selective estrogen receptors ligands and modulators. Certain drugs can have many different estrogenic effects depending on the tissue, cell and gene, and therefore they are called selective estrogen receptor modulators (SERMs). SERMs bind estrogen receptors, alter receptor conformation, and facilitate binding of co-regulatory proteins that activate or repress transcriptional activation of estrogen target genes. SERMs have estrogenic and/or antiestrogenic activity depending on their specific actions at the particular target tissue. Depending on a specific estrogenic function, SERMs could exhibit anything in the range of nearly complete agonist activity or antagonist activity. For example, some SERMs have the same agonist effect as estrogen in skeleton and cardiovascular systems but act as antagonists in breast and uterine tissues.

Estrogens have genomic and non-genomic mechanisms of action and these include classical nuclear estrogen receptors, estrogen membrane receptors, antioxidant activities, membrane fluidity effects, and effects on antiapoptotic proteins and growth factors (Cyr M, et al. Curr Pharm Des 2000; 6:1287-312; Osborne C K, et al. J Clin Oncol 2000; 18:3172-86). EFR agents, including SERMs, could modulate any or all of these estrogenic mechanisms of action.

Some EFR agents may also meet criteria defining aromatase inhibitor. For example, phytoestrogens such as from the chemical class isoflavones, may support some estrogen functions when at sufficiently high tissue concentrations. They may also inhibit aromatase activity at this same, higher or lower concentration. If the phytoestrogen is used in combination with a stronger aromatase inhibitor, then it will function at the tissue site as an EFR agent. When the human or animal is exposed to it as a single agent, it can function as an aromatase inhibitor or EFR agent, depending upon tissue concentrations, functional targets and conditions. Specific aromatase substrates (estrogen precursors) would not be effective EFR agents, unless they had inherent estrogenic functional properties that existed independently of their conversion to an estrogen by the aromatase enzymatic activity. However, if these aromatase substrates were able to successfully compete (either by higher active site affinity or higher local target tissue concentration) with the aromatase inhibitor(s) for the enzyme active site, they may then circumvent the aromatase inhibition, and they then would be able to be classified as EFR agents. Prodrugs that are metabolized via a nonaromatase pathway into a compound with EFR properties, can also be used as EFR agents. Examples include compounds that are acted upon in vivo by such enzymes reactions as hydrolysis, (de)hydroxylation, oxidation, reduction, sulfotransferase, (de)methylation, (de)lipidation, (de)prenylation, (de)glycosylation, (de)glucuronidation, (de)acetylation, (de)phosphosphorylation, (de)hydration, encapsulation, digestion and cellular transport. The compound can be a “caged-precursor” which is a chemical structure that undergoes transformation when triggered by a stimulus such as light or bioelectrical activity. The compound may be produced de novo in a protected compartment implanted within the human or animal.

Examples of EFR Agents Include but are not Restricted to:

Estradiol, ethinyl estradiol, estradiol valerate, estradiocypionate, estrone, estriol, estetrol, estropipate, 2-methoxyestradiol, hydroxyestrones, sodium estrone sulfate, equine estrogens, equilenin, equilin, PREMARIN(; conjugated estrogens, esterified estrogens, micronized estrogens, synthetic estrogens, nonsteroidal estrogens; phytoestrogens such as isoflavonoids, flavonoids, lignans, coumestan, and other natural compounds derived from plants such as soya, tea, fruits and vegetables (Jefferson W N, Newbold R R. Nutrition 2000; 16:658-62; Mazur W, Adlercreutz H. Nutrition 2000; 16:654-8); synthetic phytoestrogen ipriflavone; genistein, daidzein, enterolactone; selective estrogen receptors ligands and modulators factors (Cyr M, et al. Curr Pharm Des 2000; 6:1287-312; Osborne C K, et al. J Clin Oncol 2000; 18:3172-86) such as raloxifene, tamoxifen, indenoindoles, and estrogen partial agonist/antagonists; catechol estrogens and their metabolites such as 2-hydroxyestrone, 2-hydroxyestradiol and their 4-hydroxy isomers; 2,3-estrogen o-quinone, diethylstilbestrol, nitro-estrogens, catechol estrogen 3,4-quinone, estrophilin, formatrix, methallenestril, quinestrol, chlorotrianisene, norethisterone, norethindrone, 17-alpha-ethynyl-19-nortestosterone; dienestrol, norethynodrel, promethestrol, mestranol, tamoxifen, hydroxytamoxifen, clomiphene, chlorotrianisene, nafoxidine, hexestrol, niifepristone, RU 486; bisphenol A, p-tert-octylphenol and other endocrine disruptors; B-ring homologated estradiol analogues (Wang Z, et al. J Med Chem 2000; 43:2419-29); Estrogen Receptor Elements such as Estrogen Receptor Activation Factor, Activated Estrogen Receptor complex, and Heat Shock Protein. (see National Library of Medicine MeSH Index for “Estrogen”).

Method: Replacement Combination Therapy

For the purpose of this invention, the aromatase inhibitor(s) that cause estrogen deficit in the organism, its stereoisomers, or pharmaceutically acceptable salt are administered by various formulations and routes of administration. Similarly, the associated EFR agent(s), its stereoisomers, or pharmaceutically acceptable salts can be administered by various formulations and routes of administration. These formulations include but are not restricted to, pulmonary and nasal inhalation formulations, oral formulations, parenteral injectable and infusable formulations including intravenous, intramuscular, intradermal, subcutanous, and depot injections, and transdermal or rectal formulations. The dosage of the aromatase inhibitor compounds would vary with the particular condition being treated, the severity of the condition, the duration of the treatment, the administration route, the specific compound(s) being employed, and the patient being treated. The agents can be dosed continuously, discontinuously, as a single dose, multiple dosing frequency, chronically, acutely or any combination of these. EFR agents(s) can be given along with the aromatase inhibitor(s) or administered separately. EFR agent(s) can be administered simultaneously or disjoint in time, preceding or succeeding the administration of the aromatase inhibitor. EFR agent can be given for more, less or the same duration of time as the aromatase inhibitor agent. Several different EFR agents administered through similar or different routes of administration can be given simultaneously, or disjoint in time, for the purpose of replacing selective missing estrogen functions associated with the exposure to aromatase inhibitor. If the aromatase inhibitor exposure is unintentional and unregulated, such as from an enviromental contaminant or from an addictive substance, then the EFR agent(s) could be dosed to adjust to the schedule of administration and dosage of the unintentional and unregulated compound(s) causing the aromatase inhibition.

Method: Dosage Determination

The dosage of the aromatase inhibitor compounds may vary with the particular patient and condition being treated, the severity of the condition, the duration of the treatment, the administration route and the specific compound(s) being employed. If the aromatase inhibitor exposure is nonintentional, such as with tobacco smoke, then the compound's dosage and exposure duration can be assessed to estimate pharmacodynamic effect on aromatase and thus, the consequential estrogen deficit to be replaced. Similarly, the dosage of the EFR agent(s) will vary with the particular patient and condition being treated, the severity of the condition, the duration of the treatment, the administration route and the specific compound being employed.

The EFR agent(s) component would be dosed to provide sufficient biological activity for the desired estrogen function at the tissue target while in the presence of, or subsequent to exposure to, the aromatase inhibitor. The EFR agent(s) component may be administered with the intent to provide biological availability at the tissue target at a local concentration that would, minimally, meet the EC50 value (half-maximal efficacy concentration) for the desired estrogenic function, as determined from an examination of dose-response. When not available from a direct measurement of dose-response experiments of the desired function, the EC50 value may be estimated from assays of the binding affinity of estrogen receptors found in the targeted tissues. The target concentration for the EFR agent can be estimated, using appropriate quantitative assays of biological fluids, from monitoring the blood/plasma/serum concentration of the dosed EFR agent in the individual patient or from an in vivo, in situ, in vitro or virtual simulation of a comparable biological situation. The EFR agent may express a combination of partial agonist and partial antagonist function for the desired estrogenic activity. This can occur when a racemic mixture of stereoisomers is tested in a dose-response experiment. Weak (less potent) estrogenic compounds can also have both partial agonist and partial antagonist characteristics. These weak estrogenic compounds may act as an antagonist at the aromatase enzyme site and cause enzyme inhibition. However, when this form of aromatase inhibition occurs in the setting of a combination therapy containing a more potent aromatase inhibitor, then the aromatase inhibitory properties of the weak EFR agent are irrelevant and redundant to that of the stronger inhibitor already present. In this situation, only the selective estrogen agonist functions of the weak EFR agent would manifest at the site of action.

Formulations and Configurations of the Invention

The invention reduced to practice can include a formulation or configuration containing EFR agent(s) alone or EFR agent(s) in combination with the aromatase inhibitor component. The EFR agent(s) and the aromatase inhibitor(s) can be co-formulated or formulated separately. They may be administered together or administered separately, in time and space. In addition to administering to humans, the invention can be administered to animals. The compositions could be administered to animals in their feed, in pill form, or any other appropriate dosage forms pertinent to such animals. Examples of possible formulations, compositions, preparations and configurations follow.

Oral Formulations: Any biologically-acceptable oral dosage form well known to persons of ordinary skill in the art, and any combinations thereof, can be considered. Examples of potential dosage forms include, but are not limited to: chewable tablets, quick-dissolve tablets, effervescent tablets, reconstitutional powders, elixirs, liquids, solutions, suspensions, emulsions, tablets, caplets, multilayer-tablets, bi-layer tablets, capsules, soft gelatin capsules, hard gelatin capsules, lozenges, chewable lozenges, beads, powders, granules, particles, microparticles, dispersible granules, cachets, nutriceuticals, cereals, health bars, candies, suckers, lollipops, gums, flakes, slurries, gelatins, soups, teas, extracts, drinks and creams. The formulations may be combinations of dosage forms to create specially-timed release of drug substances and formulation components. These include immediate-release, extended-release, timed-release, sustained-release, zero-order release, osmotic-release and delayed-release, whose long-acting characteristics and combinations thereof are performed using well known procedures and techniques available to the ordinary artisan.

Inhaled Formulations: Any biologically-acceptable inhaled dosage form well known to persons of ordinary skill in the art, and any combinations thereof, can be considered. These include inhaled powders, inhaled mists, aerosol inhalants, nebulized aerosol, pump sprays, positive-pressure sprays, electrostatic sprays, aromas, pheromones, candles, perfumes, cigarettes, cigars, and pipes.

Parenteral Formulations: Any biologically-acceptable parenteral dosage form well known to persons of ordinary skill in the art, and any combinations thereof, can be considered. Examples of potential dosage forms include but are not limited to, solutions, suspensions, emulsions, boluses, intramuscular injections, polymers, microspheres, liposomes, latex beads, oils, and needleless-delivery formulations such as Powderjet. These may be administered through intravenous, intramuscular, intradermal, subcutaneous, intrauterine and peritoneal sites/routes. Practioners may utilize fiberoptic surgical tools and/or medically appropriate catheters for delivery to sites.

Depot Parenteral Formulations and Implants: Any biologically-acceptable depot parenteral dosage form well known to persons of ordinary skill in the art, and any combinations thereof, can be considered. Depots can be composed of biocompatible polymers, matrices, microspheres, proteins, lipids, nucleic acid, and biochip devices. These may be administered through or implanted any anatomical site including, but not restricted to: blood vessels, brain, eye, internal organs, heart, lung, kidney, intestines, pancreas, spleen, muscle, dermis, subdermis, uterus, peritoneal cavity, bone or periosteal surface of bone. Practioners may utilize fiberoptic surgical tools and/or medically appropriate catheters for delivery to sites.

Transdermals and Topicals: Any biologically-acceptable topical dosage form well known to persons of ordinary skill in the art, and any combinations thereof, can be considered. These may include but are not restricted to, solution, soap, oil, ointment, lotion, gel, cream, polymer or matrix. When formulated the active compound(s) may be admixed with a suitable carrier which is compatible with human skin or mucosa and which enhances transdermal penetration of the compound through the skin or mucosa. Suitable carriers are known in the art. The carrier may also include various additives commonly used in ointments and lotions and well known in the cosmetic and medical arts. For example, fragrances, antioxidants, perfumes, gelling agents, thickening agents such as carboxymethylcellulose, surfactants, stabilizers, emollients, coloring agents and other similar agents may be present.

A transdermal patch may be used to deliver the EFR agent(s), with or without the aromatase inhibitor agent(s), in accordance with known techniques. It is typically applied for a period of e.g., 1 to 4 days, but typically contacts active ingredient to a smaller surface area, allowing a slow and constant delivery of active ingredient. A number of transdermal drug delivery systems that have been developed, and are in use, are suitable for delivering the active ingredient of the present invention. The rate of release is typically controlled by a matrix diffusion, or by passage of the active ingredient through a controlling membrane. The transdermal patch device may be any of the general types known in the art including adhesive matrix and reservoir-type transdermal delivery devices. The device may include drug-containing matrixes incorporating fibers that absorb the active ingredient and/or carrier. In a reservoir-type device, the reservoir may be defined by a polymer membrane impermeable to the carrier and to the active ingredient.

In a transdermal device, the device itself maintains active ingredient in contact with the desired localized skin surface. In such a device, the viscosity of the carrier for active ingredient is of less concern than with a cream or gel. A solvent system for a transdermal device may include, for example, oleic acid, linear alcohol lactate and dipropylene glycol, or other solvent systems known in the art. The active ingredient may be dissolved or suspended in the carrier. For attachment to the skin, a transdermal patch may be mounted on a surgical adhesive tape having a hole punched in the middle. The adhesive is preferably covered by a release liner to protect it prior to use. Typical material suitable for release includes polyethylene and polyethylene-coated paper, and preferably silicone-coated for ease of removal. For applying the device, the release liner is peeled away and the adhesive attached to the patient's skin.

Transdermal formulations could also be delivered via electroporation or with ultrasound stimulation.

Spray Preparations: Any biologically-acceptable spray dosage form well known to persons of ordinary skill in the art, and any combinations thereof, can be considered. These include formulations appropriate for topical pump sprays, positive pressure sprays, and electrostatic drug sprays.

Douche and Enema Preparations: Any biologically-acceptable douche or rectal dosage form well known to persons of ordinary skill in the art, and any combinations thereof, can be considered. These include compositions appropriate for intravaginal, intrarectal, or intraurethral administration.

Suppositories: Any biologically-acceptable suppository dosage form well known to persons of ordinary skill in the art, and any combinations thereof, can be considered. These include compositions appropriate for intravaginal, cervical, intrauterine, intrarectal, or intraurethral administration.

Ophthalmic Preparations: Any biologically-acceptable ophthalmic dosage form well known to persons of ordinary skill in the art, and any combinations thereof, can be considered. These include compositions appropriate for extra or intraorbital administration. Dosage form may be applied as ointment, drops, patch, adhesive, spray or injection.

Intraoral or Intranasal Preparations: Any biologically-acceptable intranasal or intraoral dosage form well known to persons of ordinary skill in the art, and any combinations thereof, can be considered. These include compositions appropriate for the site and may be applied as ointment, drops, patch, adhesive, spray or injection. Compositions may be placed on mucosal surface or implanted at periosteal surface of bone or tooth.

Intrathecal Preparations: Any biologically-acceptable intrathecal parenteral dosage form well known to persons of ordinary skill in the art, and any combinations thereof, can be considered. These include compositions appropriate for the site and may be given through epidural, spinal or brain administration. Preparation may be solid, solution, suspension, depot or implantable device(s). Practioners may utilize fiberoptic surgical tools and/or medically appropriate catheters for delivery to sites.

Medical Devices: Implantable biological chips may be engraved, inlaid or overlaid with components. Silicon, or any biocompatible material can be used. Devices may contain nucleic acid, protein, cellular or chemical substances, singly or in combination. Biologically compatible pumps may be considered and these include infusion pumps and their individual components, for intravenous, subcutaneous, intrathecal, intragastric, intraintestinal, intrauterine, intrathoracic and intrapulmonary delivery of desired component. Pumps may have both external (ex vivo) or internal (in vivo) components. In vivo components may include catheters. Intravaginal and intrauterine drug delivery devices well known in the art can be used. Practioners may utilize fiberoptic surgical tools and/or medically appropriate catheters for delivery to sites

Biological Formulations: Biological tissues, transgenic tissues, stem cells, genetically-altered cells, cell suspension, tissue cultured cells, proteins, nucleic acids, glycoproteins or combinations thereof, may be considered as components to the invention. Active ingredients may be combined with or conjugated to biological tissues and products. They may be altered and modified from their natural states as needed for therapeutic and manufacturing goals. These biologic components include: transplanted animal and human cells and tissues (both self and nonself), antibodies, humanized monoclonals, recombinantly-expressed proteins and peptides, protein-nucleic acid combinations, encapsulated biologicals, biologicals growing in fiber optics, biologicals growing on permeable membranes, human and animal blood products, vaccines, and biosensor combination devices. They may also be bacterial, viral or plasmid or a combination thereof. They may be suspended within liposomes, or loaded into cells for subsequent therapeutic delivery and effect.

Combinations of All the Above: The invention is not restricted to a single compound or a single-route of administration. The EFR agent(s) may or may not be delivered and dosed together with the aromatase inhibitor(s) that causes the estrogen deficit. There is no limit placed as to the number of components that can be combined to deliver the desired selective estrogen function replacement to the tissue target.

Kit, Labeling and Instructions for Use: The invention will be packaged in forms well known to persons of ordinary skill in the art, and any combinations thereof, can be considered appropriate for the invention. These forms may include but are not restricted to, boxes, bottles, jars, packets, envelopes, blister packs, syringes, bags, pumps, inhaler devices, tubes, patches, stickers, spray bottles, injector pens, and boxes. The invention will be distributed as a kit in an appropriate container. The kit will contain instructions for use appropriate to the user and health practitioner. The package and kit may contain trademark names and designs appropriate to the invention.

### EXAMPLES

The following examples are further illustrations of applications of preferred embodiments of the inventive subject matter to particularly affected patients and clinical conditions, and are not to be construed as limiting the inventive subject matter thereto.

Vaginal and Vulvar Topical Preparations for the Treatment of Vulvovaginal Candidiasis

Antifungals are given to treat vulvovaginal candidiasis. However, the imidazole antifungal agents such as ketoconazole, buconazole, itraconazole and miconazole, inhibit local aromatase enzymatic conversion of estrogen precursors to estrogens as a side effect of the therapy. In addition, these antifungal drug products usually are not the subject's sole exposure to aromatase inhibiting substances. They are often taken along with concomitant medications, food stuffs and xenobiotics. The enzyme inhibition induced from exposure to the antifungal is can be additive to the aromatase inhibition arising from xenobiotics such as tobacco smoke (Osawa Y, et al. J Enzyme Inhib 1990; 4:187-200) and flavonoids (Mak P, et al. Environ Health Perspect 1999; 107:855-60; Paakki P, et al. Environ Health Perspect 2000; 108:141-5; Akbarsha M A, et al. J Reprod Fertil 2000; 120:385-390), oral contraceptives (Osawa Y, Yarborough C. Science 1982; 215:1249-51; Yamamoto T, et al. Eur J Endocrinol 1994; 130:634-40). and oral hypoglycemics of the thiazolidinediones class (Mu Y M, et al. Biochem Biophys Res Commun 2000; 271:710-3).

Vaginal, vulvar, cervical and genitourinary tissues need estrogenic presence in their cellular environments in order to allow cellular proliferation and the healing of mucosal, skin and genitourinary lesions associated with the pathogenic yeast infection. The adverse effects profile of imidazole and triazole antifungal treatment support the diagnosis of estrogen deficiency produced from their treatment. The clinical data from the vaginal antifungal product groups showed an increase in puritic vaginal irritation and headache, relative to the vehicle placebo groups (TERAZOL( Product Label, PDR 2000) despite a demonstrated reduction in vaginal cultures of Candida species.

Antifungal agents are often given to women and animals while pregnant. In primate pregnancy the fetal-placental unit becomes the primary source of estrogen production in the human pregnancy, overtaking ovarian steroidogenic function. A very recent study attempting to dissect out the critical roles of estrogen in fetal maintenance in pregnant baboons, reported that exposure to an experimental and highly selective aromatase inhibitor during pregnancy lead to a 50-70% incidence in fetal loss. The critical role for estrogen was established by administering estradiol to the drug group, which suffered no pregnancy losses (Albrecht E D, et al. Am J Obstet Gynecol 2000; 182:432-8). Instillation of antifungal agents into the vaginal vault is likely to lead to significant exposure of the pregnant uterus, placenta and fetus to these aromatase inhibitors. There are no data from well-controlled prospective studies of the outcome of topical or systemic imidazole antifungal treatments for vulvovaginal candidiasis in human pregnant subjects, although epidemiologic studies suggest clinically significant adverse outcomes consistent with those seen in animal studies. Rosa et al. (Obstet Gynecol 1987; 69:751-5) studied pregnancy outcome data from the Michigan Medicaid Prescription database after first-trimester exposure to vaginitis drug therapies. Using three separate analyses, miconazole exposure consistently showed a relative risk for spontaneous abortion of 1.4 (95% CI 1.2-1.5) that was independent of the drug therapy indication. This increased relative risk was also noted with the imidazole antifungal clotrimazole, but not with the nonimidazole antifungal agent, nystatin. Placental aromatase-produced estrogen from fetal-adrenal androgens increases steadily throughout late pregnancy and is important for initiating the onset of labor and partuition that concludes pregnancy at term (Nathanielsz P W, et al. Nat Med 1998; 4:456-9). Inhibition of placental aromatase by antifungals in late pregnancy could therefore increase the time needed to reach partuition. The prolongation of gestation that was seen in Hungarian epidemiological studies investigating human use of fluconazole as an increase in mean gestational age in the drug-exposed group (Czeizel A E, Rockenbauer M. Paediatr Perinat Epidemiol 1999; 13:58-64) is consistent with nonhuman primate data. An additional risk from exposure to aromatase inhibitors in pregnancy involves the male fetus. In late pregnancy, exposure to aromatase inhibitors may interfere with brain gender differentiation associated with CNS aromatization of fetal androgens during fetal and postnatal critical periods (Mathias L J, et al Proc Soc Exp Biol Med 1999; 221:126-30; Veney S L, et al. Neuroreport 2000; 11:3409-12).

In a preferred embodiment, the invention would combine a topical estradiol cream with the topical imidazole antifungals used to treat vulvovaginal candidiasis. EFR agent(s) used in combination with imidazole antifungal therapies would replace the missing tissue estrogen and therefore enhance vaginal mucosal cell proliferation, vaginal mucosal healing, and urethral healing (Cardozo L, et al. Obstet Gynecol 1998; 92:722-7; Samsioe G. Am J Obstet Gynecol 1998; 178:S245-9; Smith P. Acta Obstet Gynecol Scand Suppl 1993; 157:1-26). EFR agent(s) would increase vaginal secretion acidity which then could inhibit pathogenic yeast growth, and enhance the growth of nonpathogenic microbial flora, such as lactobacillus. Growth of lactobacillus bacteria enhances the ability of the vaginal tissues to fight off pathogenic bacteria (Caillouette J C, et al. Am J Obstet Gynecol 1997; 176:1270-53; Boskey E R, et al. Infect Immun 1999; 67:5170-5) reducing the incidence of bacterial vaginosis and its associated adverse events, such as risk of premature labor in pregnant infected mothers (Saling E. J Perinat Med 1998; 26:466-8; Riedewald S, et al. J Perinat Med 1990; 18:181-6). Vaginal mucosal lesions and alkaline pH are also implicated as portals of entry in HIV infection and infection of other Sexual Transmitted Diseases, so accelerated healing would diminish these risks, as well (Olinger et al. AIDS 1999; 13:1905-12; Cohen C R, et al. AIDS 1995; 9:1093-7).

The feasibility of this preferred embodiment of the invention was initially investigated in early pilot clinical investigation with individual human patients. The inventor assembled the invention from commercially-available components. The inventor prescribed the experimental off-label use of the combination of ESTRACE® estradiol 0.01% vaginal cream and MONISTAT® 2% miconazole cream to select individual human adult female patients who were experiencing relapsing symptomatic vaginal candidiasis. These patients all reported rapid resolution of their symptoms and reduction in relapses with use of this embodiment of the invention. This early unblinded patient symptom data was collected by the inventor from the period of 1988 through [symbol] invention utilized 100 mg and 200 mg miconazole suppositories in combination with 50 micrograms of estradiol vaginal cream, applied each night for 3 to 7 days. Endpoints for evaluation of invention's efficacy included vaginal pH, vaginal estrogen index, yeast culture,, symptom duration and frequency, relapse of symptoms, and recurrence of infection and symptoms. However US FDA estrogen class labeling restrictions have so far limited the scope of use of the invention in US FDA approvable clinical trials, to testing only hypogonadal postmenopausal women who have no current exposure to hormone replacement. In particular, current US FDA policy states that no women of childbearing potential can receive exogenous estrogens.

Menopause

Local conversion of androgens to estrogens by tissue aromatase is a primary source of estrogen in postmenopausal aging women. EFR agents are currently used in perimenopausal and menopausal women to prevent and/or treat vaginal atrophy, hypogonadism, diminished libido and to relieve vasomotor symptoms, urogenital atrophy, osteoporosis, alopecia and other symptoms and signs associated with menopause. Therefore, aromatase inhibitor exposure such as occurs from therapeutics, contaminants and tobacco products in this patient population increases the likelihood of adverse events associated with estrogen deficiency, further emphasizing the need for combination therapy of EFR agents with aromatase inhibitor therapeutics when used in peri- and post-menopausal women.

Endometrial Bleeding

In women with blood clotting disorders, inhibition of aromatase could result in insufficient local tissue estrogen production to support the hemostasis of the endometrial lining. When such patients are exposed to an aromatase inhibitor exposure such as from therapeutics, contaminants and tobacco products, an estrogenic agent should be given to enhance endometrial proliferation in order to mend the tissue site of bleeding and stop hemorrhage. Such patient may have liver disease, hemophilias, platelet dysfunction, blood dyscrasias, autoimmune diseases, bone marrow suppression or renal disease as the cause of their failure to maintain hemostasis.

Contraceptives

The oral contraceptive component, norethindrone (17 alpha-ethynyl-19-nortestosterone) is an irreversible inhibitor of aromatase (Osawa Y, Yarborough C. Science 1982; 215:1249-51; Yamamoto, et al. Eur J Endocrinol 1994; 130:634-40). Use of this compound may inadvertantly cause local estrogen depletion at tissue sites that usually generate local estrogen with tissue aromatase enzyme from circulation precursors. Its use may cause reduction in estrogen-induced vasodilation, contributing to cerebrovascular events, migraine or thrombotic disorders. Its use may lead to adverse changes in vaginal secretions, flora and healing. Therefore, norethindrone's efficacy may be improved by combining it with estrogenic agents targeted to provide sufficient hormone to particular areas of estrogen depletion.

Male Infertility

Spermatogenesis requires aromatase-produced estrogens as a paracrine factor. The identification of estrogen receptors and aromatase within various cell types in the testis, indicates that estrogens exert paracrine actions within the testis to promote spermatogenesis (Ebling F J, et al. Endocrinology 2000; 141:2861-9; Janulis L, et al. J Androl 1998; 19:65-71). In the male, estrogen is also the main regulator of the gonadal-pituitary feedback for the gonadotropin axis (Nauras N, et al. J Clin Endocrinol Metab 2000; 85:2370-7). Therefore, inhibitors of aromatase could contribute to male infertility. EFR agents would be used to prevent or replace the resultant estrogen deficit in the target tissues of men. One example of the invention, is the combination of an EFR agent with the antifungal compound used to treat inguinal fungal infections in order to prevent reduction/dysfunction in spermatogenesis during the treatment. Another invention would combine EFR agents with antifungals used in the treatment of the breeding aspect of the domestic animals such as race horses, dogs and beef cattle.

Cardiovascular Disease

Local conversion of androgens to estrogens by tissue aromatase is a source of estrogen for vascular dilation (especially coronary vasodilation) in, not only women, but also men. In subjects under aromatase inhibitor exposure such as from therapeutics, contaminants and tobacco products, they may lose the beneficial vasodilatative effects of local de novo estradiol synthesis that occurs in vascular endothelial cells, especially those in coronary and cerebral arteries (Harada N, et al. Circ Res 1999; 84:1285-91; Geary G G, et al. Am J Physiol Heart Circ Physiol 2000; 279:H511-9; Geary G G, et al. Am J Physiol Heart Circ Physiol 2000;279:H610-8; Mishra S K, et al. Cardiovasc Res 2000; 46:539-46; Nonaka A, et al. Invest Ophthalmol Vis Sci 2000; 41:2689-96). Therefore, patients needing aromatase inhibiting therapies who are at risk for cardiovascular, cerebrovascular and peripheral vascular disease may especially benefit from an estrogenic agent that is given in combination with the product.

Heart Failure

Supplemental estrogen replacement therapy is associated with a reduction in both overall and cardiac mortality in women>50 years of age with congestive heart failure (Reis S E, et al. J Am Coll Cardiol 2000; 36:529-33). Estrogen may be effective in heart failure because of its vasodilatory properties (Rosenfeld C R, et al. Am J Physiol Heart Circ Physiol 2000; 279:H319-28; Simoncini T, Genazzani A R. J Clin Endocrinol Metab 2000; 85:2966-9), its ability to inhibit cytokines, or because of its atheroprotective effects. Elderly women with congestive heart failure who need therapies with aromatase inhibiting effects, would benefit from receiving estrogenic agents in combination with the aromatase inhibitor.

Breast Cancer

Aromatase inhibitors are used to diminish the production of estrogens at the site of cancerous breast tissue. These agents are usually given systemically and the production of estrogen is reduced throughout the body. Selective EFR agents, such as raloxifene, can be combined with the aromatase inhibitor therapy to reduce the adverse effects of estrogen-depletion, such as effects on bone resorption and cardiovascular disease, without stimulating the growth of otherwise estrogen-sensitive breast cancer cells. Estradiol metabolites may be beneficial as an EFR agent in tumor therapy (Lippert T H, et al. Steroids 2000; 65:357-69).

Prostate Cancer

Aromatase inhibitors are used to diminish the production of estrogens at the site of cancerous or hyperplastic prostate tissue. These agents are usually given systemically and the production of estrogen is reduced throughout the body. Selective EFR agents (such as raloxifene) could be added to the therapy to reduce the effects of estrogen-depletion on bone resorption and cardiovascular disease, without stimulating prostate cancer cells.

Neurologic Diseases

The Central Nervous System (CNS), especially male brain tissue, has high rates of aromatase activity. This activity is apparent in the fetus and throughout postnatal, juvenile and adult life (Pinckard K L, et al. Domest Anim Endocrinol 2000; 18:83-96). Numerous reports consistently establish the potency of estrogens to modulate brain function of dopaminergic, cholinergic, GABAergic, glutamatergic and serotonergic neurotransmission through estrogen-mediated mechanisms and demonstrate their implications in schizophrenia and depression. Studies using in vivo and in vitro models, as well as epidemiological data, suggest that estrogens provide neuroprotection of CNS cells implicated in the etiology of neurodegenerative disorders such as Alzheimer's and Parkinson's diseases (Janowsky J S, et al. J Cogn Neurosci 2000; 12:407-14). Drugs with estrogen activity in the brain may have therapeutic potential either by modulating brain neurotransmitter transmission or through neuroprotective activity (Cyr M. Curr Pharm Des 2000; 6:1287-312). Estrogen modulates the dopaminergic system (Arvin M, et al. Brain Res 2000; 872:160-71). Low-dose estrogen is a safe and effective adjunct therapy to existing antiparkinsonian treatment in reducing motor disability in postmenopausal women with Parkinson's Disease associated with motor fluctuations (Tsang K L, et al. Neurology 2000; 54:2292-8). Estrogen deprivation leads to death of dopamine cells in the brain (Leranth C, et al. J Neurosci 2000; 20:8604-8609). Functions that depend upon aromatase conversion of substrates to estrogens, could be replaced with estrogenic agents when anti-aromatase therapies are given. These EFR agents could be given through a CNS reservoir or a CNS-implanted device when local selective CNS effect is desired or in situations when EFR agents are unable to cross the blood brain barrier when administered orally, transdermally, or parenterally.

Osteoporosis

Estrogen plays a major role in bone mineral homeostasis, maintaining a balance between bone formation and bone resorption in, not only women, but also men. Extraglandular aromatization of circulating androgen is the major source of estrogen in both post-menopausal women and men. Bone tissue itself, is an extraglandular source of local estrogen which plays an important role in bone mineral metabolism through autocrine and paracrine actions (Shozu M, Simpson E R. Mol Cell Endocrinol 1998; 139:117-29; Oz O K, et al. J Bone Miner Res 2000; 15:507-14). Serum adrenal androgen is converted to estrogen in the osteoblast and is important in maintaining bone mineral density in the postmenopausal woman (Nawata H, et al. J Steroid Biochem Mol Biol 1995; 53:165-74). Women with rheumatic diseases, especially when using corticosteroids, are in a high risk of osteoporotic fractures and atherosclerotic disease, which cause significant morbidity and mortality in later life (Julkunen H. Scand J Rheumatol 2000; 29:146-53). Estrogen therapy has alleviating effects on nighttime back pain and functional back disability in slim osteopenic premenopausal women (Kyllonen E S, et al. Spine 1999; 24:704-8). Persons at increased risk of osteoporotic bone fractures include fair-skinned or lightweight persons, smokers, heavy drinkers, persons on prolonged corticosteroid therapy, and those with early menopause or rheumatoid arthritis (Saville P D. Postgrad Med 1984; 75:135-8, 142-3).

Diabetic Nephropathy

Postmenopausal women with type 2 diabetes, hypertension and nephropathy show improved renal microvascular function when treated with estrogen agents as compared to those who are not. They have reduced mean 24-hour urine protein excretion, increased creatinine clearance, improved fasting plasma glucose, and improved serum total cholesterol (Szekacs B, et al. BJOG 2000; 107:1017-21). In such patients, aromatase inhibitor exposure, such as from contaminants, therapeutics and tobacco products, should be combined with EFR agents in order to decrease renal damage. Estrogens effects on bone turnover would also help renal disease associated osteodystrophy (Molaison E F. J Ren Nutr 2000; 10: 154-7).

Lipid Disorders

Patients with lipid disorders, often associated with diabetes, obesity and cardiovascular disease, are particularly susceptible to adverse effects from aromatase inhibition. The recent series of papers describing the aromatase knock-out mouse phenotype (Jones M E, et al. Proc Natl Acad Sci USA 2000; 97:12735-40; Nemoto Y, et al. J Clin Invest 2000; 105:1819-25) report the role of estrogen in lipid beta-oxidation and in maintaining hepatic lipid homeostasis. In addition, the oral antiglycemic agents from the thiazolidinediones class also show aromatase inhibition (Mu Y M, et al. Biochem Biophys Res Commun 2000; 271:710-3) hence adding to the block of this critical endocrine function in the diabetes disease population.

Cigarette Smoking

Tobacco smoke contains compounds that can inhibit aromatase activity. Smoking is associated with disruptions in gonadal steroid production, birth anomalies, pregnancy complications, osteoporosis, breast cancer, cardiovascular disease, peripheral and cerebrovascular disease. These complications of smoking habit may be associated with the inhibition of aromatase. Therefore, this population may benefit from a combination of EFR agent(s) to coincide with or follow the exposure to cigarette smoke. One example of such an invention could be an EFR agent released from the filter or mouthpiece of the cigarette. The cigarette could incorporate a phytoestrogen component, such as from soy extracts, that also confers antioxidant properties.

Acne, Hirsuitism and Alopecia

Studies of skin hair follicles reveal the presence of aromatase enzyme, especially in women (Sawaya M E, Price V H. J Invest Dermatol 1997; 109:296-300). If inhibition of aromatase activity occurred at the hair follicle, it may lead to virilization of the hair pattern, such as androgenic pattern alopecia or virilized facial hair growth (hirsuitism). If skin aromatization were inhibited, then local concentrations of androgens may increase and stimulate sebaceous glands to oversecrete, contributing to acne exacerbations. These complications have been reported in clinical trials of aromatase inhibiting antifungal and oncologic agents (Goss P E et al. Clin Cancer Res 1995; 1:287-94; Stevens D A, et al. Chemotherapy 1997; 43:371-7; Sugar A M, et al. Antimicrob Agents Chemother 1987; 31:1874-8). Aromatase inhibitor use should be accompanied by EFR agents to avoid these complications.

Impregnated Catheters

Chronically indwelling catheters for central venous access, intrathecal drainage, urinary bladder access, pleural drainage, colostomy drainage, or gastric/intestinal feedings, may be impregnated with an antifungal agent to suppress fungal growth on the indwelling medical device. The tissue surrounding the catheter may subsequently be deprived of locally-produced estrogen. For the blood vessels, this could lead to vaso-occlusion and thrombotic events. For the urinary catheter, this could lead to exaggerated urethral maceration and delayed healing. For the brain, this could lead to neurodegeneration or other changes in CNS function. Therefore, these devices would be less harmful and more efficacious if an EFR agent was combined with the use of the device.

While the invention has been described in detail, and with reference to specific embodiments thereof, it will be apparent to one of ordinary skill in the art that various changes and modifications can be made therein without departing from the spirit and scope thereof. Such modifications are intended to fall within the scope of the appended claims. Various publications are cited herein, the disclosures of which are incorporated by reference in their entireties.

What is claimed is:

1. A composition for administering to a subject prior to, concurrent with, and/or subsequent to, exposure to one or more inhibitors of aromatase, said composition comprising one or more estrogen function replacement, EFR, agent.

2. The composition of claim 1, wherein said estrogen function replacement agent can partially or completely replace a role of estrogen in said subject wherein said estrogen is a product of aromatase, such as estradiol or estrone.

3. The composition of claim 1, wherein said EFR agent is chosen from the group consisting of:
(i) prodrugs that are metabolized into an active agent in vivo by such enzymes reactions as: hydrolysis, dehydroxylation, hydroxylation, oxidation, reduction, sulfotransferase, methylation, demethylation, lipidation, delipidation, prenylation, deprenylation, glucosylation, deglycosylation, glucuronidation, deglucuronidation, acetylation, deacetylation, phosphorylation, dephosphosphorylation, hydration, dehydration, encapsulation, digestion and targeted cellular transport;
(ii) a caged-precursor, a chemical structure that undergoes transformation when triggered by a stimulus such as light or bioelectrical activity;
(iii) a compound produced de novo in a protected compartment implanted within the human or animal;
(iv) a racemic mixture of stereoisomers;
(v) a biological product such as a peptide, a protein, an oligonucleotide sequence, a protein-nucleic acid complex, a cell suspension, a cell tissue, a polymer-tissue matrix, a liposomal or cell organelle complex, a recombinant gene expression product, a viral or a bacterial product;
(vi) a full estrogen receptor agonist such as estradiol;
(vii) a partial estrogen receptor agonist;
(viii) a combination of partial agonists and partial antagonists;
(ix) a SERM such as indenoindoles, raloxifene, tamoxifen, benzo[a]carbazoles;
(x) a phytoestrogen such as, alpha-naphthoflavone, flavonoids, genistein, daidzein, enterolactone, ipriflavone;
(xi) an endocrine disruptor such as, p-tert-octylbutanol, DDT, polycyclic aromatic hydrocarbons, PCBs, Bisphenol A and various pesticides; and
(xii) an activated signal transduction receptor element such as, heat shock protein and estrogen receptor-ligand complex.

4. The composition of claim 1, wherein said aromatase inhibitor is defined as an agent that can partially or completely inhibit the activity of aromatase enzyme in said subject.

5. The composition of claim 1, wherein said aromatase inhibitor exposure to said subject may be intentional, unintentional, or unavoidable.

6. The composition of claim 1, wherein said aromatase inhibitor is
(i) any combination of chemical, drug, biologic, botanical product, herb supplement, vitamin supplement, dietary supplement, food product, food toxin, bacterial or viral product, air contaminant, water contaminant, or drug contaminant
(ii) prodrugs that are metabolized into an active agent in vivo by such enzymes reactions as: hydrolysis, dehydroxylation, hydroxylation, oxidation, reduction, sulfotransferase, methylation, demethylation, lipidation, delipidation, prenylation, deprenylation, glucosylation, deglycosylation, glucuronidation, deglucuronidation, acetylation, deacetylation, phosphorylation, dephosphosphorylation, hydration, dehydration, encapsulation, digestion and targeted cellular transport;
(iii) a caged-precursor, a chemical structure that undergoes transformation when triggered by a stimulus such as light or bioelectrical activity;
(iv) a compound produced de novo in a protected compartment implanted within the human or animal;
(v) a racemic mixture of stereoisomers;
(vi) a biological products such as peptide, protein, oligonucleotide sequence, protein-nucleic acid complex, cell suspension, cell tissue, polymer-tissue matrix, liposomal or cell organelle complex, recombinant gene expression product, viral or bacterial product;
(vii) 4-hydroxyandrostenedione, 4-OHA;
(viii) an endocrine disruptor such as, p-tert-octylbutanol, DDT, polycyclic aromatic hydrocarbons, PCBs, Bisphenol A and various pesticides;
(ix) norethisterone/norethindrone (17alpha-ethynyl-19-nortestosterone);
(x) a 13-retro-antiprogestin;
(xi) aminoglutethimide;
(xii) testololactone;
(xiii) an azole derivatives such as: anastrozole, fadrozole, letrozole, vorozole, roglethimide, atamestane, exemestane, formestane, YM-511(4-[N-(4-bromobenzyl)-N-(4cyanophenyl)amino]-4H-1,2,4-triazole), ZD-1033 (arimedex), NKS-01 (14-alpha-hydroxyandrost-4-ene-3,6,17-trione, ketoconazole, bifonazole, clotrimazole, econazole, isoconazole, miconazole, tioconazole, voriconazole, 4(5)-imidazoles;
(xiv) midazolam;
(xv) a vegetable, plant leaf, flower, bark or fruit;
(xvi) a synthetic flavonoid, alpha-naphthoflavone;
(xvii) a naturally-occurring flavonoid such as, chrysin, flavone, genistein, 4′-methyl ether, and Biochanin A;
(xviii) an insulin sensitizer such as troglitazone; and
(xix) a tobacco leaf, a smoke extract, tobacco juice, tobacco smoke contaminated environment, tobacco-derived gum, tobacco-derived nasal inhalant, tobacco-derived food, tobacco-derived tea, tobacco-derived drink, tobacco-derived lozenge and tobacco-derived transdermal product.

7. The composition of claim 1, wherein the formulation of said composition comprises:
(i) EFR agent alone;
(ii) EFR agent in combination with an aromatase inhibitor component; and
(iii) EFR agent and aromatase inhibitor co-formulated.

8. The composition of claim 1, wherein the formulation of said composition is:
(i) a biologically-acceptable oral dosage form such as chewable tablets, quick-dissolve tablets, effervescent tablets, reconstitutional powders, elixirs, liquids, solutions, suspensions, emulsions, tablets, caplets, multilayer-tablets, bi-layer tablets, capsules, soft gelatin capsules, hard gelatin capsules, lozenges, chewable lozenges, beads, powders, granules, particles, microparticles, dispersible granules, cachets, nutriceuticals, cereals, health bars, candies, suckers, lollipops, gums, flakes, slurries, gelatins, soups, teas, extracts, drinks and creams;
(ii) a biologically-acceptable dosage formulation for specially-timed release of drug substances and formulation components such as immediate-release, extended-release, timed-release, sustained-release, zero-order release, osmotic-release and delayed-release;
(iv) a biologically-acceptable inhaled dosage form such as inhaled powders, inhaled mists, aerosol inhalants, nebulized aerosol, pump sprays, positive-pressure sprays, electrostatic sprays, aromas, pheromones, candles, perfumes, cigarettes, cigars, and pipes;
(v) a biologically-acceptable parenteral dosage form such as solutions, suspensions, emulsions, boluses, intramuscular injections, polymers, microspheres, liposomes, latex beads, oils, and needleless-delivery formulations such as Powderjet;
(vi) a biologically-acceptable depot parenteral dosage form such as depots composed of biocompatible polymers, matrices, microspheres, proteins, lipids, nucleic acid, and biochip devices;
(vii) a biologically-acceptable topical dosage form such as solution, soap, oil, ointment, lotion, gel, cream, polymer or matrix;
(viii) a biologically-acceptable transdermal patch dosage form such as adhesive matrix and reservoir-type transdermal delivery devices;
(ix) a biologically-acceptable transdermal device dosage form such as devices with solvent systems comprising oleic acid, linear alcohol lactate and dipropylene glyco;
(x) a biologically-acceptable spray dosage form such as formulations appropriate for topical pump sprays, positive pressure sprays, and electrostatic drug sprays;
(xi) a biologically-acceptable douche or rectal dosage form such as compositions appropriate for intravaginal, intrarectal, or intraurethral administration;
(xii) a biologically-acceptable suppository dosage form such as compositions for intravaginal, cervical, intrauterine, intrarectal, or intraurethral administration;
(xiii) a biologically-acceptable ophthalmic dosage form such as compositions for extra or intraorbital administration, ointments, drops, patches, adhesives, sprays, injections, depots or implants;
(xiv) a biologically-acceptable intranasal or intraoral dosage form such as ointment, drops, patch, adhesive, spray or injection;
(xv) a biologically-acceptable intrathecal parenteral dosage form such as solids, solutions, suspensions, depots or implantable devices;
(xvi) a biologically-acceptable medical device such as devices containing singly or combinations of implantable biological chips, nucleic acids, proteins, cellular or chemical substances, and/or biosensor combination devices;
(xvii) a biologically-compatible pump device such as infusion pumps and their individual components, for intravenous, subcutaneous, intrathecal, intragastric, intraintestinal, intrauterine, intrathoracic and intrapulmonary delivery of desired component;
(xviii) a biologically-acceptable intravaginal and intrauterine drug delivery devices;
(xix) a biologically-acceptable biological product such as active ingredients combined with or conjugated to biological tissues and products;
(xx) any biologically-acceptable biological product that may be altered and modified from original natural states as needed for therapeutic and manufacturing goals, such as products suspended within liposomes, products loaded into cells, products loaded into human and animal tissues, transgenic tissues, stem cells, genetically-altered cells, cell suspensions, tissue cultured cells, proteins, nucleic acids, glycoproteins, transplanted animal and human cells and tissues, both self and nonself, antibodies, humanized monoclonals, recombinantly-expressed proteins and peptides, protein-nucleic acid combinations, encapsulated biologicals, biologicals growing in fibers, biologicals growing on permeable membranes, human and animal blood products, vaccines, bacteria, viruses or plasmids; and
(xxi) a combination of any of the formulations listed in (i) through (xx) above.

9. The composition of claim 1 wherein the package for said composition comprises:
(i) boxes, bottles, jars, packets, envelopes, blister packs, syringes, bags, pumps, inhaler devices, tubes, patches, stickers, spray bottles, injector pens;
(ii) an associated container kit appropriate for mode of distribution; and
(iii) instructions for use appropriate to the user and health practitioner.

10. A method for alleviating adverse side effects and/or enhancing the beneficial efficacy of an aromatase inhibitor in a subject, wherein said method comprises administering a combination of one or more aromatase inhibitor according to claim 6 with one or more EFR agent according to claim 3.

11. The method of claim 10 wherein said administration is simultaneous or disjoint in time, preceding or succeeding the administration of said aromatase inhibitor and said EFR agent and said aromatase inhibitor are administered continuously, discontinuously, as a single dose, with multiple dosing frequency, chronically, acutely or any combination thereof.

12. The method according to claim 10 wherein the EFR agent is administered for more, less or the same duration as said aromatase inhibitor.

13. The method according to claim 10 wherein the dosage of said aromatase inhibitor is varied to correspond with the particular patient and condition being treated, the severity of the condition, the duration of the treatment, the administration route and the specific compound being employed.

14. The method of claim 10 wherein the administration is chosen from the group consisting of: intrathecal, epidural, spinal, intravenous, inhalation, oral, topical, ophthalmic, intraorbital, extraorbital, mucosal, intravaginal, vulvar, rectal, intrauterine, peritoneal, intrathoracic, intrapulmonary, intragastric, intraintestinal, inhaled, intranasal, buccal, sublingual, parenteral, depot, intramuscular, subcutaneous, periosteal and subdermal, transdermal, and by catheter.

15. The method of claim 10 wherein dosage of EFR agent can be adjusted to unintentional and unregulated aromatase inhibitor exposure occuring from an enviromental contaminant or from an addictive substance; and dosage of aromatase inhibitor and exposure duration can be assessed to estimate pharmacodynamic effect on aromatase, and thus, estimate the consequential estrogen deficit needed to be replaced by said EFR agent

16. The method of claim 10 wherein said EFR agent are dosed to provide biological availability at the target tissue at a concentration that would, minimally, meet the EC50 value for the desired estrogen function, while in the presence of the identified aromatase inhibitor and wherein said EC50 value may be determined from an examination of dose-response data in assays of the estrogen function.

17. The method of claim 10 wherein said EFR agent are dosed to provide biological availability at the target tissue at a concentration that would, minimally, meet the EC50 value for the desired estrogen function, while in the presence of the identified aromatase inhibitor andwherein said EC50 value may be estimated from assays of the binding affinity of estrogen receptors found in similar targeted tissues.

18. The method of claim 10 wherein said EFR agent are dosed with the goal to provide biological availability at the target tissue at a concentration that would, minimally, meet the EC50 value for the desired estrogen function, while in the presence of the identified aromatase inhibitor and wherein the ideal target concentration for said EFR agent may be estimated from monitoring the blood/plasma/serum concentration of said EFR agent after dosing in the individual patient using suitable assays of biological fluids, or from an in vivo, in situ, in vitro or virtual simulation of pharmacokinetic and pharmacodynamic data of a comparable physiological situation.

19. The method of claim 10 wherein the subjects to be treated are suffering from side effects and reduced therapeutic benefit of compositions comprising an aromatase inhibitor administered as a therapeutic for a disease state or clinical indication, wherein said composition is chosen from the group consisting of:
(i) topical imidazole and triazole antifungal preparations for vaginal, vulvar, inguinal and skin treatments;
(ii) oral antifungal agents used for long term treatment of such infections as nail fungal infections, oropharyngeal and esophageal candidiasis, histoplasmosis, blastomycosis, cryptocococus, coccidioides and tuberculosis;
(iii) intravenous antifungal agents given to immunocompromised patients, such as those with AIDs, undergoing cancer chemotherapy or bone marrow transplant or those with selective immunodeficiency syndromes and hematologic diseases;
(iv) intravenous and intrathecal antifungal agents given to patients with fungal meningitis or brain abscess;
(v) chemotherapies for breast cancer and for prostate cancer;
(vi) psychotropic drugs such as midazolam;
(vii) contraceptive hormones, such as norethindrone (17 alpha-ethynyl-19-nortestosterone), an irreversible inhibitor of aromatase;
(viii) herbal and plant supplements including Over-the-Counter products and prescription botanical products;
(ix) tobacco smoke exposure as occurs in nicotine-addicted subjects and especially pregnant nicotine-addicted subjects; and
(x) impregnated catheters such as chronically indwelling catheters for central venous access, intrathecal drainage, urinary bladder access, pleural drainage, colostomy drainage, or gastric/intestinal feedings, that may be impregnated with an antifungal agent to suppress fungal growth on the indwelling medical device.

20. The method according to claim 19 wherein said disease states and clinical indications to be treated are chosen from the group consisting of:
(i) perimenopause or menopause, to prevent and/or treat vaginal atrophy, urogenital atrophy, hypogonadism, diminished libido, vasomotor symptoms, osteoporosis, and mood disturbances;
(ii) pregnancy, to prevent fetal loss and dysfunctional parturition;
(iii) cardiovascular, cerebrovascular and peripheral vascular disease, to reduce stroke, myocardial infarctions and gangrene;
(iv) heart failure, to reduce or prevent complications and mortality;
(v) male infertility, to prevent reduction or dysfunction in spermatogenesis;
(vi) breast, endometrial or prostatic cancer or hyperplasia, to prevent diseases and symptoms associated with estrogen deficit;
(vii) neurodegenerative disease, to ameliorate symptoms and reduce tissue damage;
(viii) neurodevelopment, to ameliorate symptoms and reduce tissue damage;
(ix) rheumatic disease, in osteopenic premenopausal women, fair-skinned or lightweight persons, smokers, heavy drinkers, menopausal and perimenopausal women, to prevent or reduce symptoms and complications associated with osteoporosis;
(x) diabetic nephropathy, to reduce renal complications and loss of renal function;
(xi) diabetes or a lipid disorders, to reduce or prevent complications such as atherosclerosis and other cardiovascular syndromes;
(xii) endometrial bleeding, to reduce or prevent bleeding complications and hemorrhage;
(xiii) exposure to tobacco smoke, to reduce or prevent complications associated with tobacco smoking such as intrauterine growth retardation and other pregnancy complications, cardiovascular disease, hypertension, peripheral vascular disease, accelerated skin aging, wrinkling, and headaches;
(xiv) exposure to contraceptive hormones, to reduce drug-associated complications such as migraine, vaso-occlusive disorders, thrombotic events, vaginal infections, and vaginal symptoms; and
(xv) acne, hirsuitism and alopecia, to relieve these complications.
